(19) United States
(12) Patent Application Publication
(10) Pub. No.: US 2006/0270650 A1
(43) Pub. Date: Nov. 30, 2006

(54) Combination therapy for the treatment of obesity

(76) Inventors: Tanya MacNeil, Westfield, NJ (US); Douglas J. MacNeil, Westfield, NJ (US)

Correspondence Address: MERCK AND CO., INC, P O BOX 2000, RAHWAY, NJ, 07065-0907, US

(21) Appl. No.: 11/429,721

(22) Filed: May 8, 2006

Related U.S. Application Data
(60) Provisional application No. 60/684,704, filed on May 26, 2005.

(51) Int. Cl. A61K 31/506; A61K 31/497; A61K 31/4747; A61K 31/4025; A61K 31/445; A61K 31/4412; A61K 31/19; A61K 31/397

(52) U.S. Cl. 514/210.01; 514/255.05; 514/256; 514/278; 514/326; 514/409; 514/345; 514/557

(57) ABSTRACT

The present invention relates to compositions comprising calcium and/or xanthan gum and an anti-obesity agent, useful for the treatment and prevention of diabetes, obesity and obesity-related disorders. The present invention further relates to methods of treating or preventing obesity and obesity-related disorder in a subject in need thereof by administering a composition of the present invention. The present invention further provides for pharmaceutical compositions, medicaments, and kits useful in carrying out these methods.

### BACKGROUND OF THE INVENTION

Obesity is a major health concern in Western societies. It is estimated that about 97 million adults in the United States are overweight or obese. Epidemiological studies have shown that increasing degrees of overweight and obesity are important predictors of decreased life expectancy. Obesity causes or exacerbates many health problems, both independently and in association with other diseases. The medical problems associated with obesity, which can be serious and life-threatening, include hypertension; type 2 diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arterioscelerosis; heart disease; abnormal heart rhythms; and heart arrythmias (Kopelman, P. G., Nature 404, 635-643 (2000)). Obesity is further associated with premature death and with a significant increase in mortality and morbidity from stroke, myocardial infarction, congestive heart failure, coronary heart disease, and sudden death.

Obesity is often treated by encouraging patients to lose weight by reducing their food intake or by increasing their exercise level and therefore increasing their energy output. A sustained weight loss of 5% to 10% of body weight has been shown to improve the co-morbidities associated with obesity, such as diabetes and hypertension, and can lead to improvement of obesity-related conditions such as osteoarthritis, sleep apnea and pulmonary and cardiac dysfunction.

Weight loss drugs that are currently used in monotherapy for the treatment of obesity have limited efficacy and significant side effects. Studies of the weight loss medications orlistat (Davidson, M. H. et al. (1999) JAMA 281:235-42), dexfenfluramine (Guy Grand, B. et al. (1989) Lancet 2:1142-5), sibutramine (Bray, G. A. et al. (1999) Obes. Res. &:189-98) and phentermine (Douglas, A. et al. (1983) Int. J. Obes. 7:591-5) have demonstrated a limited weight loss of about 5%-10% of body weight for drug compared to placebo. In particular, both sibutramine and orlistat reduce body weight less than 10% over a 6 month or a 1 year period. The side effects of these drugs and anti-obesity agents further limit their use. Dexfenfluramine was withdrawn from the market because of suspected heart valvulopathy; orlistat is limited by gastrointestinal side effects; the use of topiramate is limited by central nervous system effects; and the use of sibutramine is limited by its cardiovascular side effects which have led to reports of deaths and its withdrawal from the market in Italy.

Combination therapy is more likely to achieve the desired medical benefits without the trial and error involved in prescribing each agent individually during primary care. Commercially available combination therapies, which include phentermine as one of the components, have lead to mixed results. Phentermine was prescribed with fenfluramine (Pondimin®) or dexfenfluramine (Redux®) as a combination therapy known as fen-phen, which was withdrawn from the market in 1997 based on studies suggesting that the drugs cause damage to the mitral valve of the heart and pulmonary hypertension. Additionally, both fenfluramine and phentermine work through the same mechanism, namely the serotonin and norepinephrine pathway.

Due to the side effects and limited efficacy of the anti-obesity drugs currently available for mono- and combination therapy, there is a need for a combination weight loss treatment with enhanced efficacy and fewer undesirable side effects. The instant invention addresses this problem by providing a combination therapy comprised of calcium or xanthan gum and an anti-obesity agent useful in the treatment and prevention of obesity and obesity-related disorders.

Treatment with a combination of calcium and/or xanthan gum and an anti-obesity agent will lead to a larger decrease in appetite and food intake than treatment with the anti-obesity agent alone. Treatment with the compositions of the present invention allows the use of the maximum efficacious dose of calcium and/or xanthan gum, which has no significant side effects, and a sub-clinical dose of a second anti-obesity agent, with known side effects, resulting in effective treatment with fewer side effects than current monotherapies.

It is an object of the present invention to identify compositions comprising calcium and/or xanthan gum and an anti-obesity agent useful for the treatment of obesity and obesity-related diseases. It is another object of the invention to identify methods of treating and preventing obesity. It is a further object of the present invention to provide a method of manufacture of a medicament useful in the treatment of obesity.

### SUMMARY OF THE INVENTION

The present invention provides compositions comprising calcium and/or xanthan gum and an anti-obesity agent useful in the treatment or prevention of obesity, and obesity-related disorders. The present invention is further concerned with compositions comprising calcium and/or xanthan gum and an anti-obesity agent selected from the group consisting of: a 5HT transporter inhibitor, a NE transporter inhibitor, a CB-1 antagonist/inverse agonist, a Ghrelin antagonist, a H3 antagonist/inverse agonist, a MCH1R antagonist, a MCH2R agonist/antagonist, a NPY1 antagonist, an NPY5 antagonist, leptin, a leptin derivative, an opioid antagonist, an orexin antagonist, a BRS3 agonist, a CCK-A agonist, a CNTF, a CNTF derivative, a DP-IV inhibitor, a GHS agonist, a 5HT2C agonist, a Mc4r agonist, a monoamine reuptake inhibitor, an UCP-1, 2, or 3 activator, a β3 agonist, a thyroid hormone β agonist, a PDE inhibitor, a FAS inhibitor, a DGAT1 inhibitor, a DGAT2 inhibitor, an ACC2 inhibitor, a glucocorticoid antagonist, an acyl-estrogen, a lipase inhibitor, a fatty acid transporter inhibitor, a dicarboxylate transporter inhibitor, a glucose transporter inhibitor, and a serotonin reuptake inhibitor.

The compositions of the present invention are useful in the treatment or prevention of the following obesity related disorders: overeating; bulimia; hypertension; diabetes, elevated plasma insulin concentrations; insulin resistance; dyslipidemias; hyperlipidemia; endometrial, breast, prostate and colon cancer; osteoarthritis; obstructive sleep apnea; cholelithiasis; gallstones; abnormal heart rhythms; heart arrythmias; myocardial infarction; congestive heart failure; coronary heart disease; sudden death; stroke; polycystic ovarian disease; craniopharyngioma; the Prader-Willi Syndrome; Frohlich's syndrome; GH-deficient subjects; normal variant short stature; Turner's syndrome; and other pathological conditions showing reduced metabolic activity or a decrease in resting energy expenditure as a percentage of total fat-free mass, e.g, children with acute lymphoblastic leukemia.

The present invention is also concerned with treatment of these conditions, and the use of the compositions of the present invention for manufacture of a medicament useful for treating these conditions.

The invention is also concerned with pharmaceutical compositions comprising a calcium and/or xanthan and an anti-obesity agent, as active ingredients.

The present invention is also concerned with the use of calcium and/or xanthan gum and an anti-obesity agent for the manufacture of a medicament for the treatment of obesity which comprises an effective amount of calcium and/or xanthan gum and an effective amount of an anti-obesity agent, together or separately.

The present invention is also concerned with a product containing calcium and/or xanthan and an anti-obesity agent as a combined preparation for simultaneous, separate or sequential use in obesity.

The present invention also relates to the treatment of obesity with a combination of calcium and/or xanthan gum and an anti-obesity agent which may be administered separately, and combined into a kit form. The kit, according to this invention, comprises two separate pharmaceutical compositions: a first unit dosage form comprising a prophylactically or therapeutically effective amount of calcium and/or xanthan gum, or a pharmaceutically acceptable salt thereof, and a pharmaceutically acceptable carrier or diluent in a first unit dosage form, and a second unit dosage form comprising a prophylactically or therapeutically effective amount of an anti-obesity agent, or a pharmaceutically acceptable salt thereof, and a pharmaceutically acceptable carrier or diluent in a second unit dosage form and a container.

### DETAILED DESCRIPTION OF THE INVENTION

One approach to achieve greater weight loss is to combine compounds with different in vivo mechanisms of action. The present invention combines drug therapy with nutrients to cause weight loss and a decrease in food intake. Two examples of nutrients that may lead to weight loss are calcium (Zemel et. al., Obes. Res. 2004 12:582-590) and xanthan gum (U.S. Pat. No. 5,292,534).

Dietary calcium is known to play a role in maintaining skeletal integrity, in modulating chronic disease risk, and in modulating blood pressure when combined with fruits and vegetables in hypertensive patients. Recent studies also suggests that calcium rich diets may reduce the risk of cardiovascular disease and may be useful to treat obesity (Zemel et al., Obesity Research, 12, 4 (2004).

Xanthan gum is a high molecular weight natural polysaccharide produced in a culture fermentation process by the bacterium “Xanthomonas Campestris,” which is found on cabbage plants. The bacterium is cultured in a medium containing glucose, dipotassium hydrogen phosphate, a nitrogen source and trace elements to produce the extracellular xanthan polymer. Xanthan is used as a rheology control agent in aqueous systems and as a stabilizer for emulsions and suspensions, particularly in the food and cosmetic industries. Xanthan gum is also known to retard drug release in pharmaceutical formulations and has been shown to affect weight loss (U.S. Pat. No. 5,292,534).

The present invention provides compositions comprising calcium and/or xanthan gum and an anti-obesity agent useful in the treatment or prevention of obesity and obesity-related disorders.

The methods and compositions of the present invention comprise calcium. The calcium of use in the present invention may be any form of calcium known in the art. The calcium may be calcium citrate, tricalcium citrate, calcium citrate-maleate, calcium carbonate, calcium acetate, calcium phosphate, dicalcium phosphate, tricalcium phosphate, calcium lactate, calcium gluconate, calcium gluconate/lactate, coral calcium, and calcium derived from dairy products. For convenience, the use of an orally active form of calcium is also preferred.

The methods and compositions of the present invention comprise xanthan gum. The xanthan gum of use in the present invention may be any form of xanthan gum known in the art. For convenience, the use of an orally active form of xanthan gum is also preferred.

In one embodiment of the present invention, the composition is comprised of a) a therapeutically or prophylactically effective amount of calcium, or a pharmaceutically acceptable salt thereof; b) a therapeutically or prophylactically effective amount of xanthan gum, or a pharmaceutically acceptable salt thereof, and c) a therapeutically or prophylactically effective amount of an anti-obesity agent, or a pharmaceutically acceptable salt thereof. In a class of this embodiment, the composition is comprised of a therapeutically or prophylactically effective amount of calcium, or a pharmaceutically acceptable salt thereof; and a therapeutically or prophylactically effective amount of an anti-obesity agent, or a pharmaceutically acceptable salt thereof. In another class of this embodiment, the composition is comprised of a therapeutically or prophylactically effective amount of xanthan gum, or a pharmaceutically acceptable salt thereof, and a therapeutically effective amount of an anti-obesity agent, or a pharmaceutically acceptable salt thereof.

The methods and compositions of the present invention comprise an anti-obesity agent. The anti-obesity agents useful in the compositions of the present invention include: a 5HT (serotonin) transporter inhibitor, a NE (norepinephrine) transporter inhibitor, a CB-1 (cannabinoid-1) antagonist/inverse agonist, a ghrelin antagonist, a H3 (histamine H3) antagonist/inverse agonist, a MCH1R (melanin concentrating hormone 1R) antagonist, a MCH2R agonist/antagonist, a NPY1 antagonist, a NPY5 antagonist, leptin, a leptin derivative, an opioid antagonist, an orexin antagonist, a BRS3 (bombesin receptor subtype 3) agonist, a CCK-A (cholecystokinin-A) agonist, a CNTF (Ciliary neurotrophic factor), a CNTF derivative, a GHS (growth hormone secretagogue receptor) agonist, a 5HT2C (serotonin receptor 2C) agonist, a Mc4r (melanocortin 4 receptor) agonist, a monoamine reuptake inhibitor, an UCP-1 (uncoupling protein-1), 2, or 3 activator, a β3 (beta adrenergic receptor 3) agonist, a thyroid hormone β agonist, a PDE (phosphodiesterase) inhibitor, a FAS (fatty acid synthase) inhibitor, a DGAT1 (diacylglycerol acyltransferase) inhibitor, a DGAT2 inhibitor, an ACC2 (acetyl-CoA carboxylase-2) inhibitor, a glucocorticoid antagonist, an acyl-estrogen, a lipase inhibitor, a fatty acid transporter inhibitor, a dicarboxylate transporter inhibitor, a glucose transporter inhibitor, and a serotonin reuptake inhibitor.

In another embodiment of the present invention, the anti-obesity agent is selected from the group consisting of: (1) 5HT transporter inhibitor, (2) NE transporter inhibitor, (3) CB-1 antagonist/inverse agonist, (4) Ghrelin antagonist, (5) H3 antagonist/inverse agonist, (6) MCH1R antagonist, (7) MCH2R agonist/antagonist, (8) MC4R agonist, (9) NPY1 antagonist, (10) NPY5 antagonist, (11) leptin, (12) leptin derivative, (13) opioid antagonist, (14) orexin antagonist, (15) BRS3 agonist, (16) CCK-A agonist, (17) CNTF, (18) CNTF derivative, (19) DP-IV inhibitor, (20) HS agonist, (21) 5HT2C agonist; (22) monoamine reuptake inhibitor, (23) UCP-1, 2, and 3 activator; (24) 3 agonist, (25) hyroid hormone β agonist, (26) DE inhibitor, (27) FAS inhibitor, (28) DGAT1 inhibitor, (29) DGAT2 inhibitor, (30) ACC2 inhibitor, (31) glucocorticoid antagonist, (32) acyl-estrogens, (33) lipase inhibitor; (34) fatty acid transporter inhibitor, (35) dicarboxylate transporter inhibitor, (36) glucose transporter inhibitor, and (37) serotonin reuptake inhibitor, or a pharmaceutically acceptable salt thereof.

In one class this embodiment of the present invention, the anti-obesity agent is selected from the group consisting of: (1) CB-1 antagonist/inverse agonist, (2) NPY5 antagonist, (3) MCH1R antagonist, (4) BRS3 agonist; (5) CCK-A agonist; (6) ACC2 inhibitor, (7) Mc4r agonist, and (8) DP-IV inhibitor, or a pharmaceutically acceptable salt thereof.

In another class of this embodiment, the anti-obesity agent is selected from the group consisting of: (1) CB-1 antagonist/inverse agonist, (2) NPY5 antagonist, and (3) Mc4r agonist, or a pharmaceutically acceptable salt thereof.

In a subclass of this embodiment, the NPY5 antagonists useful in the present invention is selected from the group consisting of:
(1) 3-oxo-N-(5-phenyl-2-pyrazinyl)-spiro[isobenzofuran-1(3H),4′-piperidine]-1′-carboxamide,
(2) 3-oxo-N-(7-trifluoromethylpyrido[3,2-b]pyridin-2-yl)spiro-[isobenzofuran-1(3H),4′-piperidine]-1′-carboxamide,
(3) N-[5-(3-fluorophenyl)-2-pyrimidinyl]-3-oxospiro-[isobenzofuran-1(3H),4′-piperidine]-1′-carboxamide,
(4) trans-3′-oxo-N-(5-phenyl-2-pyrimidinyl)spiro[cyclohexane-1,1′(3′H)-isobenzofuran]-4-carboxamide,
(5) trans-3′-oxo-N-[1-(3-quinolyl)-4-imidazolyl]spiro[cyclohexane-1,1′(3′H)-isobenzofuran]-4-carboxamide,
(6) trans-3-oxo-N-(5-phenyl-2-pyrazinyl)spiro[4-azaiso-benzofuran-1(3H),1′-cyclohexane]-4′-carboxamide,
(7) trans-N-[5-(3-fluorophenyl)-2-pyrimidinyl]-3-oxospiro[5-azaisobenzofuran-1(3H), 1′-cyclohexane]-4′-carboxamide,
(8) trans-N-[5-(2-fluorophenyl)-2-pyrimidinyl]-3-oxospiro[5-azaisobenzofuran-1(3H),1′-cyclohexane]-4′-carboxamide,
(9) trans-N-[1-(3,5-difluorophenyl)-4-imidazolyl]-3-oxospiro[7-azaisobenzofuran-1(3H),1′-cyclohexane]-4′-carboxamide,
(10) trans-3-oxo-N-(1-phenyl-4-pyrazolyl)spiro[4-azaisobenzofuran-1(3H),1′-cyclohexane]-4′-carboxamide,
(11) trans-N-[1-(2-fluorophenyl)-3-pyrazolyl]-3-oxospiro[6-azaisobenzofuran-1(3H),1′-cyclohexane]-4′-carboxamide,
(12) trans-3-oxo-N-(1-phenyl-3-pyrazolyl)spiro[6-azaisobenzofuran-1(3H),1′-cyclohexane]-4′-carboxamide, and
(13) trans-3-oxo-N-(2-phenyl-1,2,3-triazol-4-yl)spiro[6-azaisobenzofuran-1(3H),1′-cyclohexane]-4′-carboxamide, and pharmaceutically acceptable salts thereof.

In another sub-class of this subclass, the NPY5 antagonist is 3-oxo-N-(5-phenyl-2-pyrazinyl)-spiro[isobenzofuran-1(3H),4′-piperidine]-1′-carboxamide, or a pharmaceutically acceptable salt thereof. In another subclass of this subclass, the NPY5 antagonist is trans-3′-oxo-N-[1-(3-quinolyl)-4-imidazolyl]spiro[cyclohexane-1,1′(3′H)-isobenzofuran]-4-carboxamide, or a pharmaceutically acceptable salt thereof. In another subclass of this subclass, the NPY5 antagonist is trans-N-[5-(2-fluorophenyl)-2-pyrimidinyl]-3-oxospiro[5-azaisobenzofuran-1(3H),1′-cyclohexane]-4′-carboxamide, or a pharmaceutically acceptable salt thereof. In another sub-class of this subclass, the NPY5 antagonist is trans-N-[1-(2-fluorophenyl)-3-pyrazolyl]-3-oxospiro[6-azaisobenzofuran-1(3H),1′-cyclohexane]-4′-carboxamide, or a pharmaceutically acceptable salt thereof. In another subclass of this subclass, the NPY5 antagonist is trans-3-oxo-N-(1-phenyl-3-pyrazolyl)spiro[6-azaisobenzofuran-1(3H),1′-cyclohexane]-4′-carboxamide, or a pharmaceutically acceptable salt thereof. In another subclass of this subclass, the NPY5 antagonist is 3-oxo-N-(5-phenyl-2-pyrazinyl)-spiro[isobenzofuran-1(3H),4′-piperidine]-1′-carboxamide, or a pharmaceutically acceptable salt thereof.

The above NPY5 antagonists and their preparation are disclosed in U.S. Pat. Nos. 6,326,375; 6,335,345; and International Publication No. WO 01/14376.

In another class of the first embodiment of the present invention, the NPY5 antagonist useful in the present invention is represented by the compound of structural Formula (I): 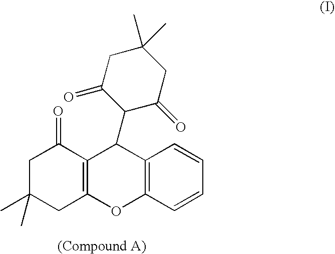
and pharmaceutically acceptable salts and tautomers thereof.

The NPY5 antagonist of Formula I (Compound A) and its preparation are disclosed in J. Organic Chemistry, vol. 31, No. 5, p. 1639 (1966); and U.S. Pat. No. 6,258,837.

In another subclass of this embodiment of the present invention, the anti-obesity agent is selected from a Mc4r agonist, or a pharmaceutically acceptable salt thereof.

In a subclass of this subclass, the Mc4r agonist is selected from the group consisting of:
(1) 2-[2-(1-{[(3S,4R)-1-tert-butyl-4-(2,4-difluorophenyl) pyrrolidin-3-yl]carbonyl}piperidin-4-yl)-5-chloro phenyl]-N-methylcarboxamide,
(2) 2-[2-(1-{[(3S,4R)-1-tert-butyl-4-(2,4-difluorophenyl) pyrrolidin-3-yl]carbonyl}piperidin-4-yl)-5-fluoro-phenyl]-N-methylcarboxamide,
(3) 2-[2-(1-{[(3S,4R)-1-tert-butyl-4-(2,4-difluorophenyl) pyrrolidin-3-yl]carbonyl}piperidin-4-yl)-5-methyl-phenyl]-N-methylcarboxamide,
(4) 2-[2-(1-{[(3S,4R)-1-tert-butyl-4-(2,4-difluorophenyl) pyrrolidin-3-yl]carbonyl}piperidin-4-yl)-phenyl]-N-methylcarboxamide,
(5) 2-[2-(1-{[(3S,4R)-1-tert-butyl-4-(2,4-difluorophenyl) pyrrolidin-3-yl]carbonyl}piperidin-4-yl)-4-methyl-phenyl]-N-methylcarboxamide,
(6) 2-[2-(1-{[(3S,4R)-1-tert-butyl-4-(2,4-difluorophenyl) pyrrolidin-3-yl]carbonyl}piperidin-4-yl)-4-fluoro-phenyl]-N-methylcarboxamide,
(7) 4-[2-(2-azetidin-1-yl-1(S)-methyl-2-oxoethyl)-4-chlorophenyl]1-{[(3S,4R)-1-tert-butyl-4-(2,4-difluorophenyl)pyrrolidin-3-yl]carbonyl}piperidine,
(8) 4-[2-(2-azetidin-1-yl-1 (R)-methyl-2-oxoethyl)-4-chlorophenyl]-1-{[(3S,4R)-1-tert-butyl-4-(2,4-difluorophenyl)pyrrolidin-3-yl]carbonyl}piperidine,
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine,
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine,
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine,
(12) 4-[2-(2-azetidin-1-yl-1-cyclopropyl-2-oxoethyl)-4-fluorophenyl]-1-{[(3S,4R)-1-tert-butyl-4-(2,4-difluorophenyl)pyrrolidin-3-yl]carbonyl}piperidine,
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide,
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide,
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide,
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide,
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl} cyclobutanecarboxamide,
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide,
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea,
(20) Methyl-2-[2-(1-{[(3S,4R)-1-tert-butyl-4-(2,4-difluoro-phenyl)pyrrolidin-3-yl]carbonyl}piperidin-4-yl)-5-chlorophenyl]-2-methylpropanoate,
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide,
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea,
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide,
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide,
(25) N-{(1S)-1-[2-(1-{[(3S,4R)-1-tert-butyl-4-(2,4-difluoro-phenyl)pyrrolidin-3-yl]carbonyl}piperidin-4-yl)-5-fluorophenyl]ethyl}acetamide,
(26) N-{(1S)-1-[2-(1-{([(3S,4R)-1-tert-butyl-4-(2,4-difluorophenyl)-pyrrolidin-3-yl]carbonyl}piperidin-4-yl)-5-chlorophenyl]propyl}acetamide, and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide, or a pharmaceutically acceptable salt thereof.

In another subclass of this embodiment of the present invention, the anti-obesity agent is selected from a cannabinoid CB₁ receptor antagonist/inverse agonist, or a pharmaceutically acceptable salt thereof.

In a subclass of this subclass, the CB₁ receptor antagonist/inverse agonist is selected from the group consisting of:
(1) N-[3-(4-chlorophenyl)-1-methyl-2-phenylpropyl]-2-(2-pyridyloxy)-2-methylpropanamide;
(2) N-[3-(4-chlorophenyl)-2-(3,5-difluorophenyl)-1-methylpropyl]-2-(2-pyridyloxy)-2-methylpropanamide;
(3) N-[3-(4-chlorophenyl)-1-methyl-2-phenyl-propyl]-2-(5-chloro-2-pyridyloxy)-2-methylpropanamide;
(4) N-[3-(4-chlorophenyl)-2-(3-pyridyl)-1-methylpropyl]-2-(5-trifluoromethyl-2-pyridyloxy)-2-methylpropanamide;
(5) N-[3-(4-chlorophenyl)-2-(3-cyanophenyl)-1-methylpropyl]-2-(5-trifluoromethyl-2-pyridyloxy)-2-methylpropanamide;
(6) N-[3-(4-chlorophenyl)-2-(5-chloro-3-pyridyl)-1-methylpropyl]-2-(5-trifluoromethyl-2-pyridyloxy)-2-methylpropanamide;
(7) N-[3-(4-chlorophenyl)-2-(5-methyl-3-pyridyl)-1-methylpropyl]-2-(5-trifluoromethyl-2-pyridyloxy)-2-methylpropanamide;
(8) N-[3-(4-chlorophenyl)-2-(5-cyano-3-pyridyl)-1-methylpropyl]-2-(5-trifluoromethyl-2-pyridyloxy)-2-methylpropanamide;
(9) N-[3-(4-chlorophenyl)-2-(3-methylphenyl)-1-methylpropyl]-2-(5-trifluoromethyl-2-pyridyloxy)-2-methylpropanamide;
(10) N-[3-(4-chlorophenyl)-2-phenyl-1-methylpropyl]-2-(4-trifluoromethyl-2-pyridyloxy)-2-methylpropanamide;
(11) N-[3-(4-chlorophenyl)-2-phenyl-1-methylpropyl]-2-(4-trifluoromethyl-2-pyrimidyloxy)-2-methylpropanamide;
(12) N-[3-(4-chlorophenyl)-1-methyl-2-(thiophen-3-yl)propyl]-2-(5-chloro-2-pyridyloxy)-2-methylpropanamide;
(13) N-[3-(5-chloro-2-pyridyl)-2-phenyl-1-methylpropyl]-2-(5-trifluoromethyl-2-pyridyloxy)-2-methylpropanamide;
(14) N-[3-(4-fluorophenyl)-2-(3-cyanophenyl)-1-methylpropyl]-2-(5-trifluoromethyl-2-pyridyloxy)-2-methylpropanamide;
(15) N-[3-(4-methoxy-phenyl)-2-(3-cyanophenyl)-1-methylpropyl]-2-(5-trifluoromethyl-2-pyridyloxy)-2-methylpropanamide;
(16) N-[3-(4-chlorophenyl)-2-(3-cyanophenyl)-1-methylpropyl]-2-(6-trifluoromethyl-4-pyrimidyloxy)-2-methylpropanamide;
(17) N-[2-(3-cyanophenyl)-1,4-dimethylpentyl]-2-(5-tri fluoromethyl-2-pyridyloxy)-2-methylpropanamide;
(18) N-[2-(3-cyanophenyl)-3-cyclobutyl-1-methylpropyl]-2-(5-trifluoromethyl-2-pyridyloxy)-2-methylpropanamide;
(19) N-[2-(3-cyanophenyl)-1-methyl-heptyl]-2-(5-trifluoromethyl-2-pyridyloxy)-2-methylpropanamide;
(20) N-[2-(3-cyanophenyl)-3-cyclopentyl-1-methylpropyl]-2-(5-trifluoromethyl-2-pyridyloxy)-2-methylpropanamide;
(21) N-[2-(3-cyanophenyl)-3-cyclohexyl-1-methylpropyl]-2-(5-trifluoromethyl-2-pyridyloxy)-2-methylpropanamide;
(22) 3-{1-[bis(4-chlorophenyl)methyl]azetidin-3-ylidene}-3-(3,5-difluorophenyl)-2,2-dimethylpropanenitrile;
(23) 1-{1-[1-(4-chlorophenyl)pentyl]azetidin-3-yl}-1-(3,5-difluorophenyl)-2-methylpropan-2-ol;
(24) 3-((S)-(4-chlorophenyl){3-[(1S)-1-(3,5-difluorophenyl)-2-hydroxy-2-methylpropyl]azetidin-1-yl}methyl)benzonitrile;
(25) 3-((S)-(4-chlorophenyl){3-[(1S)-1-(3,5-difluorophenyl)-2-fluoro-2-methylpropyl]azetidin-1-yl}methyl)benzonitrile;
(26) 3-((4-chlorophenyl){3-[1-(3,5-difluorophenyl)-2,2-dimethylpropyl]azetidin-1-yl}methyl)benzonitrile;
(27) 3-((1S)-1-{1-[(S)-(3-cyanophenyl)(4-cyanophenyl)methyl]azetidin-3-yl}-2-fluoro-2-methylpropyl)-5-fluorobenzonitrile;
(28) 3-[(S)-(4-chlorophenyl)(3-{(1S)-2-fluoro-1-[3-fluoro-5-(4H-1,2,4-triazol-4-yl)phenyl]-2-methylpropyl}azetidin-1-yl)methyl]benzonitrile; and
(29) 5-((4-chlorophenyl){3-[(1S)-1-(3,5-difluorophenyl)-2-fluoro-2-methylpropyl]azetidin-1-yl}methyl)thiophene-3-carbonitrile;
   and stereoisomers, pharmaceutically acceptable salts, hydrates, and crystalline forms thereof. The above cannabinoid CB₁ receptor antagonist/inverse agonists are disclosed in international patent publication WO 2003/077847 (published 24 Sep. 2003) and international patent publication WO 2005/000809 (published 6 Jan. 2005).

In one embodiment the cannabinoid CB₁ receptor antagonist/inverse agonist is N-[(1S,2S)-3-(4-chlorophenyl)-2-(3-cyanophenyl)-1-methylpropyl]-2-methyl-{[5-(trifluoromethyl)pyridin-2-yl]oxy}propanamide of structural formula II: 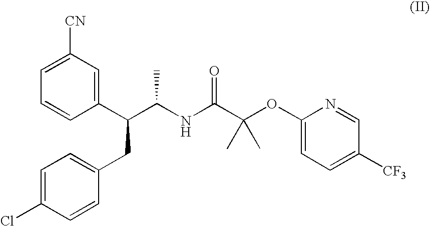
or a pharmaceutically acceptable salt, hydrate, or crystalline form thereof.

In another subclass of this class, the acyl-estrogen is selected from oleoyl-estrone, and the pharmaceutically acceptable salts thereof. In one subclass of this class, the monoamine reuptake inhibitor is selected from sibutramine, and the pharmaceutically acceptable salts thereof. In another subclass of this class, the CNTF derivative is selected from axokine, and the pharmaceutically acceptable salts thereof.

In another subclass of this class, the lipase inhibitor is selected from orlistat, and the pharmaceutically acceptable salts thereof. In another subclass of this class, the CB-1 antagonist/inverse agonist is selected from rimonabant, and the pharmaceutically acceptable salts thereof. In another subclass of this class, the anti-obesity agent is selected from leptin, and the pharmaceutically acceptable salts thereof. In another subclass of this class, the opioid antagonist is selected from nalmefene, and the pharmaceutically acceptable salts thereof. In another subclass of this class, the anti-obesity agent is selected from topiramate, and the pharmaceutically acceptable salts thereof. In yet another sub-class of this class, the anti-obesity agent is selected from metformin, and the pharmaceutically acceptable salts thereof.

In another embodiment of the present invention, the anti-obesity agent is selected from the group consisting of: a minorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate; fenfluramine, fenisorex, fenproporex, fludorex, fluminorex, furfurylmethylamphetamine, levamfetamine, levophacetoperane, mazindol, mefenorex; metamfepramone, metformin, methamphetamine; norpseudoephedrine, pentorex, phendimetrazine, phenmetrazine, phentermine, phenylpropanolamine, Phytopharm compound 57, picilorex, topiramate, and zonisamide, or a pharmaceutically acceptable salt thereof. In a class of this embodiment, the anti-obesity agent is selected from the group consisting of: dexfenfluramine, fenfluramine, phentermine, and sibutramine, or a pharmaceutically acceptable salt thereof.

Another aspect of the present invention relates to methods of treating or preventing obesity or an obesity-related disorder in a subject in need thereof by administering an effective amount of calcium and/or xanthan and an anti-obesity agent. The present invention also relates to pharmaceutical compositions, and medicaments useful for carrying out these methods.

In another embodiment of the present invention, the invention relates to a method of treating or preventing obesity in a subject in need thereof comprising administration to said subject a) a therapeutically or prophylactically effective amount of calcium, or a pharmaceutically acceptable salt thereof; b) a therapeutically or prophylactically effective amount of xanthan gum, or a pharmaceutically acceptable salt thereof; and c) a therapeutically or prophylactically effective amount of an anti-obesity agent, or a pharmaceutically acceptable salt thereof. In a class of this embodiment, the invention relates to a method of treating or preventing obesity in a subject in need thereof comprising administration to said subject a) a therapeutically or prophylactically effective amount of calcium, or a pharmaceutically acceptable salt thereof; and b) a therapeutically or prophylactically effective amount of an anti-obesity agent, or a pharmaceutically acceptable salt thereof. In another class of this embodiment, the invention relates to a method of treating or preventing obesity in a subject in need thereof comprising administration to said subject a) a therapeutically or prophylactically effective amount of xanthan gum, or a pharmaceutically acceptable salt thereof; and b) a therapeutically or prophylactically effective amount of an anti-obesity agent, or a pharmaceutically acceptable salt thereof.

In another embodiment of the present invention, the invention relates to a method of treating or preventing an obesity-related disorder in a subject in need thereof comprising administration to said subject a) a therapeutically or prophylactically effective amount of calcium, or a pharmaceutically acceptable salt thereof; b) a therapeutically or prophylactically effective amount of xanthan gum, or a pharmaceutically acceptable salt thereof; and c) a therapeutically or prophylactically effective amount of an anti-obesity agent, or a pharmaceutically acceptable salt thereof. In a class of this embodiment, the invention relates to a method of treating or preventing an obesity-related disorder in a subject in need thereof comprising administration to said subject a) a therapeutically or prophylactically effective amount of calcium, or a pharmaceutically acceptable salt thereof; and b) a therapeutically or prophylactically effective amount of an anti-obesity agent, or a pharmaceutically acceptable salt thereof. In another class of this embodiment, the invention relates to a method of treating or preventing an obesity-related disorder in a subject in need thereof comprising administration to said subject a) a therapeutically or prophylactically effective amount of xanthan gum, or a pharmaceutically acceptable salt thereof; and b) a therapeutically or prophylactically effective amount of an anti-obesity agent, or a pharmaceutically acceptable salt thereof.

In another embodiment of the present invention, the invention relates to a method of maintaining weight loss in a subject in need thereof comprising administration to said subject a) a therapeutically effective amount of calcium, or a pharmaceutically acceptable salt thereof; b) a therapeutically effective amount of xanthan gum, or a pharmaceutically acceptable salt thereof; and c) a therapeutically effective amount of an anti-obesity agent, or a pharmaceutically acceptable salt thereof. In a class of this embodiment, the invention relates to a method of maintaining weight loss in a subject in need thereof comprising administration to said subject a) a therapeutically effective amount of calcium, or a pharmaceutically acceptable salt thereof; and b) a therapeutically effective amount of an anti-obesity agent, or a pharmaceutically acceptable salt thereof. In another class of this embodiment, the invention relates to a method of maintaining weight loss in a subject in need thereof comprising administration to said subject a) a therapeutically effective amount of xanthan gum, or a pharmaceutically acceptable salt thereof; and b) a therapeutically effective amount of an anti-obesity agent, or a pharmaceutically acceptable salt thereof.

In another embodiment of the present invention, the invention relates to the use of a therapeutically effective amount of calcium and/or xanthan gum, or a pharmaceutically acceptable salt thereof; and a therapeutically effective amount of an anti-obesity agent, or a pharmaceutically acceptable salt thereof, for the manufacture of a medicament useful for the treatment or prevention of obesity in a subject in need of such treatment. In another embodiment of the present invention, the invention relates to the use of a therapeutically effective amount of calcium and/or xanthan gum, or a pharmaceutically acceptable salt thereof; and a therapeutically effective amount of an anti-obesity agent, or a pharmaceutically acceptable salt thereof, for the manufacture of a medicament useful for the treatment or prevention of an obesity-related disorder in a subject in need of such treatment.

In another embodiment of the present invention, the invention relates to the use of calcium and/or xanthan gum, or a pharmaceutically acceptable salt thereof; and an anti-obesity agent, or a pharmaceutically acceptable salt thereof; for the manufacture of a medicament for the treatment or prevention of obesity which comprises an effective amount of calcium and/or xanthan gum and an effective amount of anti-obesity agent, together or separately. In another embodiment of the present invention, the invention relates to the use of calcium and/or xanthan gum, or a pharmaceutically acceptable salt thereof; and an anti-obesity agent, or a pharmaceutically acceptable salt thereof; for the manufacture of a medicament for the treatment or prevention of an obesity-related disorder which comprises an effective amount of calcium and/or xanthan gum and an effective amount of anti-obesity agent, together or separately. In another embodiment of the present invention, the invention relates to the use of calcium and/or xanthan gum, or a pharmaceutically acceptable salt thereof; and an anti-obesity agent, or a pharmaceutically acceptable salt thereof; for the manufacture of a medicament for maintaining weight loss which comprises an effective amount of calcium and/or xanthan gum and an effective amount of anti-obesity agent, together or separately.

In another embodiment of the present invention, the invention relates to a product containing calcium and/or xanthan gum, or a pharmaceutically acceptable salt thereof, and an anti-obesity agent, or a pharmaceutically acceptable salt thereof, as a combined preparation for simultaneous, separate or sequential use in obesity. In another embodiment of the present invention, the invention relates to a product containing calcium and/or xanthan gum, or a pharmaceutically acceptable salt thereof; and an anti-obesity agent, or a pharmaceutically acceptable salt thereof; as a combined preparation for simultaneous, separate or sequential use in an obesity-related disorder. In another embodiment of the present invention, the invention relates to a product containing calcium and/or xanthan gum, or a pharmaceutically acceptable salt thereof; and an anti-obesity agent, or a pharmaceutically acceptable salt thereof; as a combined preparation for simultaneous, separate or sequential use in maintaining weight loss.

In a class of the embodiments of the present invention, the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovarian disease; craniopharyngioma; the Prader-Willi Syndrome; Frohlich's syndrome; GH-deficient subjects; normal variant short stature; Turner's syndrome; and acute lymphoblastic leukemia. In a subclass of this class, the obesity-related disorder is diabetes.

In another embodiment of the present invention, the invention relates to a kit comprising at least one unit dosage of a therapeutically or prophylactically effective amount of calcium and/or xanthan gum, or a pharmaceutically acceptable salt thereof, and at least one unit dosage of a therapeutically or prophylactically effective amount of an anti-obesity agent, or a pharmaceutically acceptable salt thereof.

Calcium useful in the present invention, includes but is not limited to, the calcium in the form of a calcium salt. Specific calcium salts useful in the combinations of the present invention, include but are not limited to, calcium citrate, tricalcium citrate, calcium citrate-maleate, calcium carbonate, calcium acetate, calcium phosphate, dicalcium phosphate, tricalcium phosphate, calcium lactate, calcium gluconate, calcium gluconate/lactate, coral calcium, and calcium derived from dairy products. In one embodiment of the present invention, the calcium salt is calcium citrate. In another embodiment of the present invention, the calcium salt is calcium carbonate.

One of ordinary skill in the art can readily identify calcium, calcium salts and calcium complexes useful in the compositions and methods of the present invention. Calcium, calcium salts and calciums complexes which are useful in the present invention are generally orally bio-available.

One of ordinary skill in the art can readily identify xanthan gum, also known as xanthan or corn sugar gum, useful in the compositions and methods of the present invention. Use of different strains or fermentation conditions may give rise to differing degrees of acetylation and pyruvylation, which moderates the functionality of the xanthan gum. Xanthan gum produced by different strains of the Xanthomonas campestris bacterium are encompassed within the scope of the present invention. Xanthan gum is available in food and technical grades (clear solution, salt tolerant, dust free, easily dispersible, high viscosity, reduced pseudoplasticity), different viscosity ranges and different particle sizes (fine, normal, coarse) all of which are encompassed within the scope of the present invention. Xanthan gum useful in the present invention is generally orally bioavailable.

As used herein, the term “anti-obesity agent” includes compounds that reduce total food intake by 5 to 30%, or reduce caloric intake or selectively reduce intake of specific components of the diet such as carbohydrates or fats by 5 to 30%; compounds which, when administered to a subject, act to increase the metabolic rate of the subject, particularly those agents which increase metabolic rate by at least 5%, preferably 10%, most preferably 20% in 24 hour energy expenditure when administered to the subject; and compounds that inhibit the absorption of 10 to 50% of the nutrients.

One of ordinary skill in the art can readily identify anti-obesity agents useful in the compositions and methods of the present invention. Anti-obesity agents that decrease food intake can be evaluated in rodents according to the procedures described in: Daniels, A. J. et al., Regulatory Peptides, 106:47-54 (2002); Halaas, J. L. et. al., Science, 269: 543-546 (1995); and Strack, A. M., Obesity Research, 10:173-81 (2002). Anti-obesity agents that increase metabolic rate are routinely evaluated in rodents (Atgie, C., Comp. Biochem. Physiol. A. Mol. Integr. Physiol. 119:629-36 (1998); Himms-Hagan, J., American J. Physiology, 266:R1371-82 (1994)), and, even when inactive in rodents, are tested in additional species such as dog and monkey before ultimately being tested in humans (Connacher, A. A. et. al., Int'l J. Obesity, 16: 685-694 (1992); Connacher, A. A. et. al., Am. J. Clin. Nutr., 55: 258S-261S (1992); Connacher, A. A. et. al., Brit. Med. J., 296: 1217-1220 (1998)). The utility of anti-obesity agents that enhance metabolic rate is supported by experiments with mice, in which the RII-beta gene has been deleted, that were shown to be resistant to diet induced obesity (D. E. Cummings et al. Nature 382: 622-626 (1996)). Anti-obesity agents that inhibit nutrient absorption can be evaluated in: Badr M. Z. and Chen, T. S., Toxicology, 34:333-40 (1985); Sorribas, V., J. Pharm. Pharmacol., 44:1030-2 (1992).

Serotonin (5HT) transport inhibitors useful in this invention include, but are not limited to, paroxetine, fluoxetine, fenfluramine, fluvoxamine, sertraline, and imipramine.

Norepinephrine (NE) transport inhibitors useful in this invention include, but are not limited to, GW 320659, despiramine, talsupram, and nomifensine.

Cannabinoid receptor 1 (CB-1) antagonist/inverse agonists useful in the present invention include, but are not limited to, rimonabant (Sanofi Synthelabo), AMT-251, and SR-14778 and SR 141716A (Sanofi Synthelabo), SLV-319 (Solvay), BAY 65-2520 (Bayer), and those disclosed in U.S. Pat. Nos. 5,532,237, 4,973,587, 5,013,837, 5,081,122, 5,112,820, 5,292,736, 5,624,941, 6,028,084, PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, WO 01/64632, WO 01/64633, WO 01/64634, WO02/076949, WO 03/007887, WO 04/048317, and WO 05/000809; and EPO Application No. EP-658546, EP-656354, EP-576357.

Dipeptidyl peptidase IV (DP-IV) inhibitors useful in the present invention include, but are not limited to, isoleucine thiazolidide, valine pyrrolidide, NVP-DPP728, LAF237, P93/01, TSL 225, TMC-2A/2B/2C, FE 999011, P9310/K364, VIP 0177, SDZ 274444; and the compounds disclosed in U.S. Pat. No. 6,699,871, which is incorporated herein by reference; International Patent Application Nos. WO 03/004498; WO 03/004496; WO 02/083128; WO 02/062764; WO 03/000250; WO 03/002530; WO 03/002531; WO 03/002553; WO 03/002593; WO 03/000180; and WO 03/000181; and European Patent Application No. EP 1 258 476.

Ghrelin antagonists useful in the present invention, include: PCT Application Nos. WO 01/87335, and WO 02/08250.

Histamine 3 (H3) antagonist/inverse agonists useful in the present invention include: PCT Application No. WO 02/15905; and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)). Specific H3 antagonists/inverse agonists useful in the present invention include, but are not limited to, thioperamide, 3-(1H-imidazol-4-yl)propyl N-(4-pentenyl)carbamate, clobenpropit, iodophenpropit, imoproxifan, and GT2394 (Gliatech).

Melanin-concentrating hormone 1 receptor (MCH1R) antagonists and melanin-concentrating hormone 2 receptor (MCH2R) agonist/antagonists useful in the present invention include PCT Patent Application Nos. WO 01/82925, WO 01/87834, WO 02/06245, WO 02/04433, and WO 02/51809; and Japanese Patent Application No. JP 13226269. Specific MCH1R antagonists useful in the present invention include, but are not limited to, T-226296 (Takeda).

Neuropeptide Y1 (NPY1) antagonists useful in the present invention, include: U.S. Pat. No. 6,001,836; and PCT Application Nos. WO 96/14307, WO 01/23387, WO 99/51600, WO 01/85690, WO 01/85098, WO 01/85173, and WO 01/89528. Specific examples of NPY1 antagonists useful in the present invention include, but are not limited to, BIBP3226, J-115814, BIBO 3304, LY-357897, CP-671906, and GI-264879A.

Neuropeptide Y5 (NPY5) antagonists useful in the present invention, include, but are not limited to, GW-569180A, GW-594884A, GW-587081×, GW-548118×, FR226928, FR 240662, FR252384, 1229U91, GI-264879A, CGP71683A, LY-377897, PD-160170, SR-120562A, SR-120819A and JCF-104, and those disclosed in U.S. Pat. Nos. 6,057,335; 6,043,246; 6,140,354; 6,166,038; 6,180,653; 6,191,160; 6,313,298; 6,335,345; 6,337,332; 6,326,375; 6,329,395; 6,340,683; 6,388,077; 6,462,053; 6,649,624; and 6,723,847, hereby incorporated by reference in their entirety; European Patent Nos. EP-01010691, and EP-01044970; and PCT International Patent Publication Nos. WO 97/19682, WO 97/20820, WO 97/20821, WO 97/20822, WO 97/20823, WO 98/24768; WO 98/25907; WO 98/25908; WO 98/27063, WO 98/47505; WO 98/40356; WO 99/15516; WO 99/27965; WO 00/64880, WO 00/68197, WO 00/69849, WO 01/09120, WO 01/14376; WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 02/48152, and WO 02/49648; WO 02/094825; WO 03/014083; Wo 03/076443, WO 03/078422, WO 03/10191; WO 03/092889; WO 04/002986; WO 04/031175; and WO 04/050652. Additional specific NPY 5 antagonists useful in the combinations of the present invention, include, but are not limited to the compounds described in Norman et al., J. Med. Chem. 43:4288-4312 (2000).

Leptin includes, but is not limited to, recombinant human leptin (PEG-OB, Hoffman La Roche) and recombinant methionyl human leptin (Amgen). Leptin derivatives (e.g., truncated forms of leptin) useful in the present invention include: Pat. Nos. 5,552,524; 5,552,523; 5,552,522; 5,521,283; and PCT International Publication Nos. WO 96/23513; WO 96/23514; WO 96/23515; WO 96/23516; WO 96/23517; WO 96/23518; WO 96/23519; and WO 96/23520.

Opioid antagonists useful in the present invention include: PCT Application No. WO 00/21509. Specific opioid antagonists useful in the present invention include, but are not limited to, nalmefene (Revex®), 3-methoxynaltrexone naloxone, and naltrexone.

Orexin antagonists useful in the present invention include: PCT Patent Application Nos. WO 01/96302, WO 01/68609, WO 02/51232, and WO 02/51838. Specific orexin antagonists useful in the present invention include, but are not limited to, SB-334867-A.

An acyl-estrogen useful in the present invention include oleoyl-estrone (del Mar-Grasa, M. et al., Obesity Research, 9:202-9 (2001)).

Cholecystokinin-A (CCK-A) agonists useful in the present invention include U.S. Pat. No. 5,739,106. Specific CCK-A agonists include, but are not limited to, AR-R 15849, GI 181771, JMV-180, A-71378, A-71623 and SR146131.

Specific ciliary neurotrophic factors (CNTF) useful in the present invention include, but are not limited to, GI-181771 (Glaxo-SmithKline); SR146131 (Sanofi Synthelabo); butabindide; PD170,292, PD 149164 (Pfizer). CNTF derivatives useful in the present invention include, but are not limited to, axokine (Regeneron); and PCT Application Nos. WO 94/09134, WO 98/22128, and WO 99/43813.

Growth hormone secretagogue (GHS) agonists useful in the present invention include: U.S. Pat. No. 6,358,951, and U.S. Patent Application Nos. 2002/049196 and 2002/022637; and PCT Application Nos. WO 01/56592, and WO 02/32888. Specific GHS agonists include, but are not limited to, NN703, hexarelin, MK-0677, SM-130686, CP-424,391, L-692,429 and L-163,255.

5HT2C agonists useful in the present invention include: U.S. Pat. No. 3,914,250; and PCT Application Nos. WO 02/36596, WO 02/48124, WO 02/10169, WO 01/66548, WO 02/44152; WO 02/51844, WO 02/40456, and WO 02/40457. Specific 5HT2C agonists useful in this invention include, but are not limited to, BVT933, DPCA37215, WAY161503, and R-1065.

Mc4r agonists useful in the present invention include, but are not limited to, Melanotan II, ME-10142, and ME-10145 (Melacure), CHIR86036 (Chiron); PT-141, and PT-14 (Palatin), and those disclosed in: U.S. Pat. Nos. 6,410,548; 6,294,534; 6,350,760; 6,458,790; 6,472,398; and 6,376,509; US Patent Publication No. US2004/009751, and PCT Application Nos. WO 99/64002; WO 00/74679; WO 01/70708; WO 01/70337; WO 01/74844; WO 01/91752; WO 01/991752; WO 02/059095; WO 02/059107; WO 02/059108; WO 02/059117; WO 02/067869; WO 02/068387; WO 02/068388; WO 02/067869; WO 02/11715; WO 02/12166; WO 02/12178; WO 03/007949; WO 03/009847; WO 04/024720; WO 04/078716; WO 04/078717; WO 04/087159; WO 04/089307; and WO 05/009950.

Monoamine reuptake inhibitors useful in the present invention include: PCT Application Nos. WO 01/27068, and WO 01/62341. Specific monoamine reuptake inhibitors useful in the present invention include, but are not limited to, sibutramine (Meridia®/Reductil®) disclosed in U.S. Pat. Nos. 4,746,680, 4,806,570, and 5,436,272, and U.S. Patent Publication No. 2002/0006964. The present invention encompasses sibutratmine as a racemic mixture, as optically pure isomers (+) and (−), or a pharmaceutically acceptable salt, solvent, hydrate, clathrate or prodrug thereof; particularly sibutramine hydrochloride monohydrate.

Serotonin reuptake inhibitors useful in the present invention include: U.S. Patent Application No. 6,365,633; and PCT Patent Application Nos. WO 01/27060, and WO 01/162341.

Uncoupling Protein (UCP-1, UCP-2, and UCP-3) activators useful in the present invention include: PCT Patent Application No. WO 99/00123. Specific uncoupling protein (UCP-1, UCP-2, and UCP-3) activators useful in the present invention include, but are not limited to, phytanic acid, 4-[(E)-2-(5,6,7,8-tetrahydro-5,5,8,8-tetramethyl-2-napthalenyl)-1-propenyl]benzoic acid (TTNPB), and retinoic acid.

β3 adrenergic receptor (β3) agonists useful in the present invention include: U.S. Pat. No. 5,705,515, and U.S. Pat. No. 5,451,677; and PCT Patent Application Nos. WO 01/74782, and WO 02/32897. Specific β3 agonists useful in the present invention include, but are not limited to, AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, and SR 59119A.

Thyroid hormone β0 agonists useful in the present invention include: PCT Application No. WO 02/15845; and Japanese Patent Application No. JP 2000256190. Specific thyroid hormone β0 agonists useful in the present invention include, but are not limited to, KB-2611 (KaroBioBMS).

Specific fatty acid synthase (FAS) inhibitors useful in the present invention, include, but are not limited to, Cerulenin and C75.

Specific phosphodieterase (PDE) inhibitors useful in the present invention, include, but are not limited to, theophylline, pentoxifylline, zaprinast, sildenafil, amrinone, milrinone, cilostamide, rolipram, and cilomilast.

Lipase inhibitors useful in the present invention include: PCT Application No. WO 01/77094. Specific lipase inhibitors useful in the present invention include, but are not limited to, orlistat (Xenical®), Triton WR1339, RHC80267, lipstatin, tetrahydrolipstatin, teasaponin, and diethylumbelliferyl phosphate.

Topiramate (Topimax®), indicated as an anti-convulsant and an anti-convulsant, has been shown to increase weight loss.

Metformin (Glucophage®) is indicated for patients with non-insulin dependent diabetes mellitus, particularly those with refractory obesity. Physician's Desk Reference® page 1080-1086, (56^{th} ed. 2002).

Specific bombesin (BRS-3) agonists useful in the present invention, include, but are not limited to, [D-Phe6,beta-Ala11,Phe13,Nle14]Bn(6-14) and [D-Phe6,Phe13]Bn(6-13)propylamide, and those compounds disclosed in Pept. Sci. 2002 August; 8(8): 461-75).

Zonisamide, a marketed antiepileptic drug with serotonergic and dopaminergic activity in addition to the ability to block sodium and calcium channels, has been shown to result in weight loss in epileptic adults and in obese adults.

The above compounds are only illustrative of the calcium, xanthan gum and anti-obesity agents that can be used in the compositions of the present invention. As this listing of compounds is not meant to be comprehensive, the methods of the present invention may employ any form of calcium, any form of xanthan gum, and any anti-obesity agent, and are not limited to any particular structural class of compounds.

The present invention also relates to the treatment of obesity with a combination of calcium and/or xanthan gum, and an anti-obesity agent which may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of calcium and/or xanthan gum, or a pharmaceutically acceptable salt thereof, and a pharmaceutically acceptable carrier or diluent in a first unit dosage form, and a second unit dosage form comprising a prophylactically or therapeutically effective amount of another anti-obesity agent, or a pharmaceutically acceptable salt thereof, and a pharmaceutically acceptable carrier or diluent in a second unit dosage form. The kit further comprises a container. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

The term “pharmaceutically acceptable salt” refers to the pharmaceutically acceptable and common salts, for example, a base addition salt to carboxyl group when the compound has a carboxyl group, or an acid addition salt to amino or basic heterocyclyl when the compound has an amino or basic heterocyclyl group, including quaternary ammonium salts, prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, trifluoro acetate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to calcium, xanthan gum, and anti-obesity agents are meant to also include the pharmaceutically acceptable salts thereof.

The pharmaceutically acceptable salts of the composition of the instant invention include the composition wherein one of the individual components of the composition is in the form of a pharmaceutically acceptable salt, or the composition wherein all of the individual components are in the form of pharmaceutically acceptable salts (wherein the salts for each of the components can be the same or different), or a pharmaceutically acceptable salt of the combined components (i.e., a salt of the composition).

The compounds in the compositions of the present invention include stereoisomers, such as optical isomers, diastereomers and geometerical isomers, or tautomers depending on the mode of substitution. The compounds may contain one or more chiral centers and occur as racemates, racemic mixtures and as individual diastereomers, enantiomeric mixtures or single enantiomers, or tautomers, with all isomeric forms being included in the present invention. The present invention is meant to comprehend all such isomeric forms of the compounds in the compositions of the present invention, and their mixtures. Therefore, where a compound is chiral, the separate enantiomers, substantially free of the other, are included within the scope of the invention; further included are all mixtures of the two enantiomers. Also included within the scope of the invention are polymorphs, hydrates and solvates of the compounds of the instant invention.

The present invention includes within its scope prodrugs of the compounds in the compositions of this invention. In general, such prodrugs will be functional derivatives of the compounds in these compositions which are readily convertible in vivo into the required compound. Thus, in the methods of treatment of the present invention, the term “administering” shall encompass the treatment of obesity and obesity-related disorders with the compounds specifically disclosed as elements of the composition or with compounds which may not be specifically disclosed, but which convert to the specified compounds in vivo after administration to the patient. Conventional procedures for the selection and preparation of suitable prodrug derivatives are described, for example, in “Design of Prodrugs,” ed. H. Bundgaard, Elsevier, 1985.

The compositions of the present invention are useful for the treatment or prevention of disorders associated with excessive food intake, such as obesity and obesity-related disorders. The obesity herein may be due to any cause, whether genetic or environmental. The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricaemia, lower back pain, gallbladder disease, gout, kidney cancer, and increased anesthetic risk. The compositions of the present invention are also useful for reducing the risk of secondary outcomes of obesity, such as reducing the risk of left ventricular hypertrophy. The compositions of the present invention are also useful to treat Alzheimer's disease.

The term “metabolic syndrome”, also known as syndrome X, is defined in the Third Report of the National Cholesterol Education Program Expert Panel on Detection, Evaluation and Treatment of High Blood Cholesterol in Adults (ATP-1). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus—type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. Another outcome of treatment may be to maintain weight loss. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Another outcome of prevention may be to prolong resistance to weight gain. Another outcome of prevention may be to prevent weight regain. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The terms “administration of” and or “administering a” compound should be understood to mean providing a compound of the invention or a prodrug of a compound of the invention to a subject in need of treatment. The instant pharmaceutical composition includes administration of a single pharmaceutical dosage formulation which contains calcium and/or xanthan gum in combination with an anti-obesity agent, as well as administration of each active agent in its own separate pharmaceutical dosage formulation. Where separate dosage formulations are used, the individual components of the composition can be administered at essentially the same time, i.e., concurrently, or at separately staggered times, i.e. sequentially prior to or subsequent to the administration of the other component of the composition. The instant pharmaceutical composition is therefore to be understood to include all such regimes of simultaneous or alternating treatment, and the terms “administration” and “administering” are to be interpreted accordingly.

Administration in these various ways are suitable for the present compositions as long as the beneficial pharmaceutical effect of the combination of the calcium and/or xanthan gum and the anti-obesity agent is realized by the patient at substantially the same time. Such beneficial effect is preferably achieved when the target blood level concentrations of each active drug are maintained at substantially the same time. It is preferred that the combination of calcium and/or xanthan gum and the anti-obesity agent be co-administered concurrently on a once-a-day dosing schedule; however, varying dosing schedules, such as the calcium and/or xanthan once, twice or three times a day and the anti-obesity agent once, twice, three or more times per day, is also encompassed herein. A single oral dosage formulation comprised of calcium and/or xanthan and an anti-obesity agent is preferred. A single dosage formulation will provide convenience for the patient, which is an important consideration especially for patients with diabetes or obese patients who may be in need of multiple medications.

The term “subject”, as used herein refers to an animal, preferably a mammal, most preferably a human, who has been the object of treatment, observation or experiment. The term “subject in need thereof” refers to a subject who is in need of treatment or prophylaxis as determined by a researcher, veterinarian, medical doctor or other clinician. In one embodiment, the subject in need of treatment is an obese mammal. In another embodiment, the subject in need of treatment is an obese human with one or more obesity-related co-morbidities. In another embodiment, the subject in need of treatment is an obese human without obesity-related co-morbidities.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated. The novel methods of treatment of this invention are for disorders known to those skilled in the art.

The term “prophylactically effective amount” as used herein means the amount of the active compounds in the composition that will elicit the biological or medical response in a tissue, system, subject, or human that is being sought by the researcher, veterinarian, medical doctor or other clinician, to prevent the onset of obesity or an obesity-related disorder in subjects as risk for obesity or the obesity-related disorder.

The magnitude of prophylactic or therapeutic dose of the active ingredients (e.g. calcium, xanthan gum, anti-obesity agent) of the composition will, of course, vary with the nature of the severity of the condition to be treated and with the particular compound in the composition and its route of administration. It will also vary according to the age, weight and response of the individual patient. In general, the daily dose range of each compound in the combination lies within the range of from about 0.0001 mg/kg to about 100 mg/kg, preferably from about 0.001 mg/kg to about 50 mg/kg body weight of a subject in single or divided doses. On the other hand, it may be necessary to use dosages outside these limits in some cases.

For use where a composition for intravenous administration is employed, a suitable dosage range is from about 0.0001 mg/kg to about 50 mg/kg, preferably from 0.001 mg/kg to about 20 mg/kg of each compound in the composition per day.

In the case where an oral composition is employed, a suitable dosage range is, e.g. from about 0.001 mg/kg to about 100 mg/kg of each compound in the composition per day, preferably from about 0.01 mg to about 7,000 mg per day. For oral administration, the compositions are preferably provided in the form of tablets containing from 0.01 mg to 3,000 mg, preferably 0.01; 0.05; 0.1; 0.2; 0.5; 1.0; 2.5; 5; 10; 15; 20; 25; 30; 40; 50; 75; 100; 125; 150; 175; 200; 225; 250; 500; 750; 850; 1,000; 1,200; 1,400; 1,500; 1,600; 1,800; 2000; 2,200; 2,400; 2,500; 2,600; 2,800; and 3,000 milligrams of each active ingredient for the symptomatic adjustment of the dosage to the subject to be treated. This dosage regimen may be adjusted to provide the optimal therapeutic response.

The compounds of this invention can be administered to humans in the dosage ranges specific for each compound. In general, for treating obesity and/or an obesity related disorder, the calcium is administered at a daily dosage of from about 0.0001 mg/kg to about 100 mg/kg of body weight orally. More specifically, when treating obesity and/or obesity related disorders generally satisfactory results may be obtained when calcium, or a pharmaceutically acceptable salt thereof, is administered at a daily oral dosage of from about 10 mg to about 2,500 mg of a day, preferably from about 400 mg to about 1,300 mg per day, given in a single dose or in divided doses two to six times a day, or in sustained release form.

In general, for treating obesity and/or an obesity related disorder, the xanthan gum is administered at a daily dosage of from about 0.0001 mg/kg to about 1000 mg/kg of body weight orally. More specifically, when treating obesity and/or obesity related disorders generally satisfactory results may be obtained when xanthan gum, or a pharmaceutically acceptable salt thereof, is administered at a daily oral dosage of from about 0.001 mg/kg to about 150 mg/kg, preferably from about 0.001 mg/kg to about 10 mg/kg of body weight, given in a single dose or in divided doses two to six times a day, or in sustained release form.

In general, for treating obesity and/or an obesity related disorder, the anti-obesity agent is administered at a daily dosage of from about 0.0001 mg/kg to about 100 mg/kg of body weight, preferably from about 0.001 mg/kg to about 50 mg/kg, given in a single dose or in divided doses two to six times per day, or in sustained release form.

In general, for treating obesity and/or an obesity related disorder, the NPY5 antagonist is administered at a daily dosage of from about 0.0001 mg/kg to about 100 mg/kg of body weight orally. More specifically, when treating obesity and/or obesity related disorders generally satisfactory results may be obtained when an NPY5 antagonist, or a pharmaceutically acceptable salt thereof, is administered at a daily oral dosage of from about 0.001 mg/kg to about 100 mg/kg, preferably from about 0.001 mg/kg to about 10 mg/kg of body weight, given in a single dose or in divided doses two to six times a day, or in sustained release form. A NPY5 antagonist of Formula I, or a pharmaceutically acceptable salt thereof, may be administered at a daily oral dosage of from about 1 mg/kg to about 30 mg/kg of body weight, given in a single dose or in divided doses two to six times a day, or in sustained release form. In general, for treating obesity and/or an obesity related disorder, the MC4R agonist is administered at a daily dosage of from about 0.0001 mg/kg to about 100 mg/kg of body weight orally. More specifically, when treating obesity and/or obesity related disorders generally satisfactory results may be obtained when an MC4R agonist, or a pharmaceutically acceptable salt thereof, is administered at a daily oral dosage of from about 0.001 mg/kg to about 100 mg/kg, preferably from about 0.001 mg/kg to about 10 mg/kg of body weight, given in a single dose or in divided doses two to six times a day, or in sustained release form. A MC4R agonist, or a pharmaceutically acceptable salt thereof, may be administered at a daily oral dosage of from about 1 mg/kg to about 30 mg/kg of body weight, given in a single dose or in divided doses two to six times a day, or in sustained release form. In general, for treating obesity and/or an obesity related disorder, the CB-1 antagonist/inverse agonist is administered at a daily dosage of from about 0.0001 mg/kg to about 100 mg/kg of body weight orally. More specifically, when treating obesity and/or obesity related disorders generally satisfactory results may be obtained when a CB-1 antagonist/inverse agonist, or a pharmaceutically acceptable salt thereof, is administered at a daily oral dosage of from about 0.001 mg/kg to about 100 mg/kg, preferably from about 0.001 mg/kg to about 10 mg/kg of body weight, given in a single dose or in divided doses two to six times a day, or in sustained release form. A CB-1 antagonist/inverse agonist of Formula II, or a pharmaceutically acceptable salt thereof, may be administered at a daily oral dosage of from about 1 mg/kg to about 30 mg/kg of body weight, given in a single dose or in divided doses two to six times a day, or in sustained release form.

Leptin may be administered at a daily dosage of from about 0.01 mg/kg to about 20 mg/kg, preferably, from about 0.01 mg/kg to about 0.3 mg/kg, preferably injected in a single dose or in divided doses.

Metformin may be administered at a daily dosage of from about 0.01 mg/kg to about 100 mg/kg, preferably from about 1 mg/kg to about 50 mg/kg in a single dose or in divided doses 2 to 3 times per day, or in sustained release form; more preferably the daily dose is 500 mg, 850 mg, 1000 mg, 1500 mg, 2000 mg or 2550 mg orally given as a single dose or in divided doses 2 to 3 times per day.

Nalmefene may be administered at a daily dosage of from about 0.0001 mg/kg to about 10 mg/kg, preferably from about 0.001 to about 0.05 mg/kg.

Orlistat may be administered at a daily dosage of from about 20 mg to about 1200 mg, preferably from about 120 mg to 400 mg in a single dose or divided doses 2 to 3 times per day or in sustained release form; more preferably a 120 mg single dose 3 times per day, or in sustained release form.

Sibutramine may be administered at a daily dosage of from about 0.01 mg/kg to about 10 mg/kg, preferably from about 0.01 mg/kg to about 1 mg/kg in a single dose or in divided doses 2 to 3 times per day, or in sustained release form; more preferably the single daily dose of sibutramine is 5 mg, 10 mg, 15 mg, 20 mg or 30 mg orally.

Rimonabant may be administered at a daily dosage of from about 0.01 mg/kg to about 8 mg/kg, more preferably from about 0.3 mg/kg to about 3 mg/kg of body weight in a single dose or in divided doses 2 to 3 times per day, or in sustained release form.

Topiramate (Topamax®) may be administered at a daily dosage of from about 10 mg to about 1,600 mg per day, preferably from about 50 mg to about 400 mg per day in a single dose or in divided doses, or in sustained release form.

Zonasamide may be administered at a daily dosage of from about 10 mg to about 1,500 mg per day, preferably from about 100 mg to about 600 mg per day in a single dose or in divided doses, or in sustained release form. More preferably zonasamide may be administered at a daily dosage of from about 100 mg/day orally, with gradual increase to 400 mg/day and further increase to 600 mg/day for patients losing less than 5% of body weight at the end of 12 weeks.

The effective dosage of each of the active ingredients employed in the composition may vary depending on the particular compound employed, the mode of administration, the condition being treated and the severity of the condition being treated. Thus, the dosage regimen utilizing the compositions of the present invention is selected in accordance with a variety of factors including type, species, age, general health, body weight, diet, sex and medical condition of the subject; the severity of the condition to be treated; the renal and hepatic function of the patient; the drug combination; and the particular compounds employed and their routes of administration. A physician, clinician or veterinarian of ordinary skill can readily determine and prescribe the effective amount of the drug required to prevent, counter or arrest the progress of the condition.

The weight ratio of calcium and xanthan gum to the anti-obesity agent may be varied and will depend upon the effective dose of each ingredient. Generally, an effective dose of each will be used. Thus, for example, when calcium citrate is combined with an anti-obesity agent, such as an NPY 5 antagonist, the weight ratio of calcium citrate to the NPY5 antagonist will generally range from about 1000:1 to about 1:1000, preferably about 200:1 to about 1:200.

Compositions of xanthan gum and an anti-obesity agent will generally also be within the aforementioned range, but in each case, an effective dose of each active ingredient should be used.

Another aspect of the present invention provides pharmaceutical compositions comprising a pharmaceutical carrier and a therapeutically effective amount of each compound in the composition of the present invention. The term “composition”, as in pharmaceutical composition, is intended to encompass a product comprising the active ingredient(s), and the inert ingredient(s), such as pharmaceutically acceptable excipients, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing calcium and/or xanthan gum, additional active ingredient(s) such as a second anti-obesity agent, and pharmaceutically acceptable excipients.

Any suitable route of administration may be employed for providing a subject, especially a human, with an effective dosage of a composition of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a combination of calcium and/or xanthan gum with an anti-obesity agent, as active ingredients or a pharmaceutically acceptable salt thereof, and may also contain a pharmaceutically acceptable carrier, and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compounds suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and seventy of the conditions being treated and on the nature of the active ingredient. These compositions may be conveniently presented in unit dosage form and prepared by any of the methods well-known in the art of pharmacy.

For administration by inhalation, the compositions of the present invention are conveniently delivered in the form of an aerosol spray presentation from pressurized packs or nebulizers. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients.

Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will be continuous rather than intermittent throughout the dosage regimen.

The compositions of the present invention can also be administered in the form of liposome delivery systems, such as small unilamellar vesicles, large unilamellar vesicles and multilamellar vesicles. Liposomes can be formed from a variety of phospholipids, such as cholesterol, sterylamine or phosphatidylcholines.

Compositions of the present invention may also be delivered by the use of monoclonal antibodies as individual carriers to which the compound molecules are coupled. The compounds in these compositions may also be coupled with soluble polymers as targetable drug carriers. Such polymers can include polyvinyl-pyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

Compositions of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

In practical use, each compound in the compositions of the present invention (e.g. calcium, xanthan, or anti-obesity agent) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredients, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil.

Desirably, each tablet contains from 0.01 to 3,000 mg, particularly 0.01 mg to 3,000 mg, preferably 0.01; 0.05; 0.1; 0.2; 0.5; 1.0; 2.5; 5; 10; 15; 20; 25; 30; 40; 50; 75; 100; 125; 150; 175; 200; 225; 250; 500; 750; 850; 1,000; 1,200; 1,400; 1,500; 1,600; 1,800; 2000; 2,200; 2,400; 2,500; 2,600; 2,800; and 3,000 milligrams of each active ingredient in the composition of the present invention (e.g., calcium, xanthan gum, anti-obesity agent) for the symptomatic adjustment of the dosage to the subject to be treated; and each cachet or capsule contains from about 0.01 to 3,000 mg, particularly 0.01 mg to 3,000 mg, preferably 0.01; 0.05; 0.1; 0.2; 0.5; 1.0; 2.5; 5; 10; 15; 20; 25; 30; 40; 50; 75; 100; 125; 150; 175; 200; 225; 250; 500; 750; 850; 1,000; 1,200; 1,400; 1,500; 1,600; 1,800; 2000; 2,200; 2,400; 2,500; 2,600; 2,800; and 3,000 milligrams of each active in the composition of the present invention (e.g., calcium, xanthan gum, anti-obesity agent) for the symptomatic adjustment of the dosage to the subject to be treated.

Exemplifying the invention is a pharmaceutical composition comprising calcium and/or xanthan gum and a second anti-obesity agent described above and a pharmaceutically acceptable carrier. Also exemplifying the invention is a pharmaceutical composition made by combining any of the calcium and/or xanthan gum and anti-obesity agents described above and a pharmaceutically acceptable carrier. An illustration of the invention is a process for making a pharmaceutical composition comprising combining any of the calcium and/or xanthan gum and anti-obesity agents described above and a pharmaceutically acceptable carrier.

The dose may be administered in a single daily dose or the total daily dosage may be administered in divided doses of two to six times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compositions of the present invention:

| Injectable Suspension (I.M.) | mg/mL | |
|---|---|---|
| Calcium citrate | 700 | |
| NPY5 antagonist of Formula I | 1 | |
| Xanthan gum | 200 | |
| Water for injection to a total volume of | 2 | mL |

| Tablet | mg/tablet |
|---|---|
| Calcium citrate | 475 |
| NPY5 antagonist of Formula I | 25 |
| Xanthan gum | 500 |
| | 1000 |
| Capsule | mg/capsule |
| Calcium citrate | 500 |
| NPY5 antagonist of Formula I | 100 |
| Xanthan gum | 400 |
| | 1000 |

It will be understood that the scope of compositions of the compounds of this invention with other agents useful for treating or preventing obesity and obesity-related conditions includes in principle any combination with any pharmaceutical composition useful for treating obesity and obesity-related disorders.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### EXAMPLE 1

In Vivo Study for Combination Therapy with a NPY5 Antagonist and Calcium or Xanthan Gum

Male C57BL/6J mice (CLEA Japan Inc., 12-16 months old at the beginning of the drug administration) are used. Mice are given water and regular pellet chow (CE-2, CLEA Japan Inc.) ad libitum. They are kept in an animal room which is maintained at 23±2° C. temperature, 55±15% relative humidity and on a 12-hr light-dark cycle (7:00-19:00) during a quarantine and acclimatization period of 1 week. Before the start of drug administration, mice are fed a MHF diet (Oriental BioService Co., Tokyo, Japan) for about 9 to 10 months until the body weight gain reaches a plateau. After the body weight gain reaches a plateau, the diet is changed to a powder MHF diet. The powder MHF diet is given by powder feeder (small dishes). Diet and dishes are changed everyday, and daily food intake is measured. During this period, animals are orally administered vehicle (0.5% methylcellulose in distilled water) by gavage once-daily. After the stable feeding is observed, the amount of new food is adjusted to daily food intake+0.3 g, to minimize the amount of spilled food. After the acclimation period, the MHF diet-fed mice are divided into four groups to match average values of body weight and food intake (n=7-10). Two of the groups are orally administered either vehicle or compound A at a dose of 100 mg/kg once-daily for 1.5 months by gavage, respectively. Another three groups are treated with vehicle or compound A and given an effective dose of calcium citrate; xanthan gum; or calcium citrate and xanthan gum. The administration is done one and half hours before the beginning of the dark period following the measurement of body weight. If the duration of action of the calcium or xanthan gum is limited, it is dosed twice-daily. The administration is done one and half hours before the beginning of the dark period following body weight measurement and one and half hours after the beginning of the light period. The amount of food is divided into two meals and given at about 8:00 and 18:00 to avoid a long duration of fasting. Food and body weight are measured.

Effective anti-obesity combinations result in a greater body weight change when the NPY5 antagonist and calcium are given together, than the body weight change seen with either compound is administered alone; or result in a greater body weight change when the NPY5 antagonist and xanthan gum are given together, than the weight change seen with either compound administered alone. Maximum body weight change may be obtained when the NPY5 antagonist, calcium, and xanthan gum are given together.

### EXAMPLE 2

Human Study for Combination Therapy with NPY5 Antagonist and Calcium or Xanthan Gum

800 people with a BMI >30 are advised to diet and increase their physical activity. After a two-week placebo run-in period, which includes a standardized program of diet, physical activity, and lifestyle changes, the patients are randomized into 7 treatment groups: placebo; an effective dose of a NPY5 antagonist, such as 1,000 mg of Compound A; an effective dose of calcium, such as 1,000 mg of calcium citrate; an effective dose of xanthan gum, such as 500 mg of xanthan; an effective dose of the NPY5 antagonist plus an effective dose of calcium; an effective dose of the NPY5 antagonist plus an effective dose of xanthan gum; and an effective dose of the NPY5 antagonist plus an effective dose of calcium and xanthan gum. The NPY5 antagonist is given in tablet form once or more per day, as previously determined to be effective. The calcium and xanthan gum are separately given in tablet form once or more per day, as previously determined to be effective. Patients are treated for 6 months, body weights are measured weekly, and appetite, hunger, satiety are measured weekly using standard questionnaires.

Effective anti-obesity combinations result in a greater body weight change when calcium and/or xanthan gum and the second anti-obesity agent are given together, than the body weight change seen with either compound is administered alone.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A composition comprising
(a) calcium, or a pharmaceutically acceptable salt thereof; and
(b) an anti-obesity agent selected from the group consisting of:
(1) 5HT transporter inhibitor,
(2) NE transporter inhibitor,
(3) CB-1 antagonist/inverse agonist,
(4) Ghrelin antagonist,
(5) H3 antagonist/inverse agonist,
(6) MCH1R antagonist,
(7) MCH2R agonist/antagonist,
(8) MC4R agonist,
(9) NPY1 antagonist,
(10) NPY5 antagonist,
(11) leptin,
(12) leptin derivative,
(13) opioid antagonist,
(14) orexin antagonist,
(15) BRS3 agonist,
(16) CCK-A agonist,
(17) CNTF,
(18) CNTF derivative,
(19) DP-IV inhibitor,
(20) GHS agonist,
(21) 5HT2C agonist;
(22) monoamine reuptake inhibitor,
(23) UCP-1, 2, and 3 activator;
(24) β3 agonist,
(25) thyroid hormone β agonist,
(26) PDE inhibitor,
(27) FAS inhibitor,
(28) DGAT1 inhibitor,
(29) DGAT2 inhibitor,
(30) ACC2 inhibitor,
(31) glucocorticoid antagonist,
(32) acyl-estrogens,
(33) lipase inhibitor;
(34) fatty acid transporter inhibitor,
(35) dicarboxylate transporter inhibitor,
(36) glucose transporter inhibitor, and
(37) serotonin reuptake inhibitor,
or a pharmaceutically acceptable salt thereof.

2. The composition of claim 1 wherein the anti-obesity agent is selected from the group consisting of:
(1) CB-1 antagonist/inverse agonist,
(2) NPY5 antagonist,
(3) MCH1 R antagonist,
(4) BRS3 agonist;
(5) CCK-A agonist;
(6) ACC2 inhibitor,
(7) Mc4r agonist, and
(8) DP-IV inhibitor,
or a pharmaceutically acceptable salt thereof.

3. The composition of claim 2 wherein the anti-obesity agent is selected from the group consisting of:
(1) CB-1 antagonist/inverse agonist,
(2) NPY5 antagonist, and
(3) Mc4r agonist,
or a pharmaceutically acceptable salt thereof.

4. The composition of claim 3 wherein the Mc4r agonist is selected from the group consisting of:
(1) N-{(1S)-1-[2-(1-{[(3S,4R)-1-tert-butyl-4-(2,4-difluorophenyl)-pyrrolidin-3-yl]carbonyl}piperidin-4-yl)-5-fluorophenyl]ethyl}acetamide,
(2) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide,
(3) N-{(S)-1-[2-(1-{([(3S,4R)-1-tert-butyl-4-(2,4-difluorophenyl)-pyrrolidin-3-yl]carbonyl}piperidin-4-yl)-5-chlorophenyl]ethyl}acetamide,
(4) 2-[2-(1-{[(3S,4R)-1-tert-butyl-4-(2,4-difluorophenyl) pyrrolidin-3-yl]carbonyl}piperidin-4-yl)-5-chloro phenyl]-N-methylcarboxamide,
(5) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide, and
(6) 4-[2-(2-azetidin-1-yl-1 (S)-methyl-2-oxoethyl)-4-chlorophenyl]-1-{[(3S,4R)-1-tert-butyl-4-(2,4-difluorophenyl)pyrrolidin-3-yl]carbonyl}piperidine,
and pharmaceutically acceptable salts thereof.

5. The composition of claim 3 wherein the NPY5 antagonist is selected from the group consisting of
(1) 3-oxo-N-(5-phenyl-2-pyrazinyl)-spiro[isobenzofuran-1(3H),4′-piperidine]-1′-carboxamide;
(2) 3-oxo-N-(7-trifluoromethylpyrido[3,2-b]pyridin-2-yl)spiro-[isobenzofuran-1(3H),4′-piperidine]-1′-carboxamide;
(3) N-[5-(3-fluorophenyl)-2-pyrimidinyl]-3-oxospiro-[isobenzofuran-1(3H),4′-piperidine]-1′-carboxamide;
(4) trans-3′-oxo-N-(5-phenyl-2-pyrimidinyl)spiro[cyclohexane-1,1′(3′H)-isobenzofuran]-4-carboxamide;
(5) trans-3′-oxo-N-[1-(3-quinolyl)-4-imidazolyl]spiro[cyclohexane-1,1′(3′H)-isobenzofuran]-4-carboxamide;
(7) trans-3-oxo-N-(5-phenyl-2-pyrazinyl)spiro[4-azaiso-benzofuran-1(3H),1′-cyclohexane]-4′-carboxamide;
(8) trans-N-[5-(3-fluorophenyl)-2-pyrimidinyl]-3-oxospiro[5-azaisobenzofuran-1(3H),1′-cyclohexane]-4′-carboxamide;
(9) trans-N-[5-(2-fluorophenyl)-2-pyrimidinyl]-3-oxospiro[5-azaisobenzofuran-1(3H),1′-cyclohexane]-4′-carboxamide;
(10) trans-N-[1-(3,5-difluorophenyl)-4-imidazolyl]-3-oxospiro[7-azaisobenzofuran-1(3H),1′-cyclohexane]-4′-carboxamide;
(11) trans-3-oxo-N-(1-phenyl-4-pyrazolyl)spiro[4-azaisobenzofuran-1(3H),1′-cyclohexane]-4′-carboxamide;
(12) trans-N-[1-(2-fluorophenyl)-3-pyrazolyl]-3-oxospiro[6-azaisobenzofuran-1(3H),1′-cyclohexane]-4′-carboxamide;
(13) trans-3-oxo-N-(1-phenyl-3-pyrazolyl)spiro[6-azaisobenzofuran-1(3H),1′-cyclohexane]-4′-carboxamide; and
(14) trans-3-oxo-N-(2-phenyl-1,2,3-triazol-4-yl)spiro[6-azaisobenzofuran-1(3H),1′-cyclohexane]-4′-carboxamide;
or a pharmaceutically acceptable salt thereof.

6. The composition of claim 3 wherein the CB₁ receptor antagonist/inverse agonist is selected from the group consisting of:
(1) N-[3-(4-chlorophenyl)-1-methyl-2-phenylpropyl]-2-(2-pyridyloxy)-2-methylpropanamide;
(2) N-[3-(4-chlorophenyl)-2-(3,5-difluorophenyl)-1-methylpropyl]-2-(2-pyridyloxy)-2-methylpropanamide;
(3) N-[3-(4-chlorophenyl)-1-methyl-2-phenyl-propyl]-2-(5-chloro-2-pyridyloxy)-2-methylpropanamide;
(4) N-[3-(4-chlorophenyl)-2-(3-pyridyl)-1-methylpropyl]-2-(5-trifluoromethyl-2-pyridyloxy)-2-methylpropanamide;
(5) N-[3-(4-chlorophenyl)-2-(3-cyanophenyl)-1-methylpropyl]-2-(5-trifluoromethyl-2-pyridyloxy)-2-methylpropanamide;
(6) N-[3-(4-chlorophenyl)-2-(5-chloro-3-pyridyl)-1-methylpropyl]-2-(5-trifluoromethyl-2-pyridyloxy)-2-methylpropanamide;
(7) N-[3-(4-chlorophenyl)-2-(5-methyl-3-pyridyl)-1-methylpropyl]-2-(5-trifluoromethyl-2-pyridyloxy)-2-methylpropanamide;
(8) N-[3-(4-chlorophenyl)-2-(5-cyano-3-pyridyl)-1-methylpropyl]-2-(5-trifluoromethyl-2-pyridyloxy)-2-methylpropanamide;
(9) N-[3-(4-chlorophenyl)-2-(3-methylphenyl)-1-methylpropyl]-2-(5-trifluoromethyl-2-pyridyloxy)-2-methylpropanamide;
(10) N-[3-(4-chlorophenyl)-2-phenyl-1-methylpropyl]-2-(4-trifluoromethyl-2-pyridyloxy)-2-methylpropanamide;
(11) N-[3-(4-chlorophenyl)-2-phenyl-1-methylpropyl]-2-(4-trifluoromethyl-2-pyrimidyloxy)-2-methylpropanamide;
(12) N-[3-(4-chlorophenyl)-1-methyl-2-(thiophen-3-yl)propyl]-2-(5-chloro-2-pyridyloxy)-2-methylpropanamide;
(13) N-[3-(5-chloro-2-pyridyl)-2-phenyl-1-methylpropyl]-2-(5-trifluoromethyl-2-pyridyloxy)-2-methylpropanamide;
(14) N-[3-(4-fluorophenyl)-2-(3-cyanophenyl)-1-methylpropyl]-2-(5-trifluoromethyl-2-pyridyloxy)-2-methylpropanamide;
(15) N-[3-(4-methoxy-phenyl)-2-(3-cyanophenyl)-1-methylpropyl]-2-(5-trifluoromethyl-2-pyridyloxy)-2-methylpropanamide;
(16) N-[3-(4-chlorophenyl)-2-(3-cyanophenyl)-1-methylpropyl]-2-(6-trifluoromethyl-4-pyrimidyloxy)-2-methylpropanamide;
(17) N-[2-(3-cyanophenyl)-1,4-dimethylpentyl]-2-(5-trifluoromethyl-2-pyridyloxy)-2-methylpropanamide;
(18) N-[2-(3-cyanophenyl)-3-cyclobutyl-1-methylpropyl]-2-(5-trifluoromethyl-2-pyridyloxy)-2-methylpropanamide;
(19) N-[2-(3-cyanophenyl)-1-methyl-heptyl]-2-(5-trifluoromethyl-2-pyridyloxy)-2-methylpropanamide;
(20) N-[2-(3-cyanophenyl)-3-cyclopentyl-1-methylpropyl]-2-(5-trifluoromethyl-2-pyridyloxy)-2-methylpropanamide;
(21) N-[2-(3-cyanophenyl)-3-cyclohexyl-1-methylpropyl]-2-(5-trifluoromethyl-2-pyridyloxy)-2-methylpropanamide;
(22) 3-{1-[bis(4-chlorophenyl)methyl]azetidin-3-ylidene}-3-(3,5-difluorophenyl)-2,2-dimethylpropanenitrile;
(23) 1-{1-[1-(4-chlorophenyl)pentyl]azetidin-3-yl}-1-(3,5-difluorophenyl)-2-methylpropan-2-ol;
(24) 3-((S)-(4-chlorophenyl){3-[(1S)-1-(3,5-difluorophenyl)-2-hydroxy-2-methylpropyl]azetidin-1-yl}methyl)benzonitrile;
(25) 3-((S)-(4-chlorophenyl){3-[(1S)-1-(3,5-difluorophenyl)-2-fluoro-2-methylpropyl]azetidin-1-yl}methyl)benzonitrile;
(26) 3-((4-chlorophenyl){3-[1-(3,5-difluorophenyl)-2,2-dimethylpropyl]azetidin-1-yl}methyl)benzonitrile;
(27) 3-((1S)-1-{1-[(S)-(3-cyanophenyl)(4-cyanophenyl)methyl]azetidin-3-yl}-2-fluoro-2-methylpropyl)-5-fluorobenzonitrile;
(28) 3-[(S)-(4-chlorophenyl)(3-{(1S)-2-fluoro-1-[3-fluoro-5-(4H-1,2,4-triazol-4-yl)phenyl]-2-methylpropyl}azetidin-1-yl)methyl]benzonitrile;
(29) 5-((4-chlorophenyl){3-[(1S)-1-(3,5-difluorophenyl)-2-fluoro-2-methylpropyl]azetidin-1-yl}methyl)thiophene-3-carbonitrile; and
(30) N-[(1S,2S)-3-(4-chlorophenyl)-2-(3-cyanophenyl)-1-methylpropyl]-2-methyl-{[5-(trifluoromethyl)pyridin-2-yl]oxy}propanamide;
and stereoisomers, pharmaceutically acceptable salts, hydrates, and crystalline forms thereof.

7. The composition of claim 3 wherein the CB-1 receptor antagonist/inverse agonist is selected from rimonabant, and the pharmaceutically acceptable salts thereof.

8. The composition of claim 1 wherein calcium is calcium citrate.

9. The composition according to claim 1 further comprising a pharmaceutically acceptable carrier.

10. The composition of claim 1 further comprising xanthan gum.

11. The use of the composition of claim 1 for the manufacture of a medicament for the treatment of obesity or an obesity-related disorder which comprises an effective amount of calcium and an effective amount of anti-obesity agent, together or separately.

12. A product containing the composition of claim 1 as a combined preparation for simultaneous, separate or sequential use in obesity or an obesity-related disorder.

13. The use of the composition of claim 10 for the manufacture of a medicament for the treatment of obesity or an obesity-related disorder which comprises an effective amount of calcium, an effective amount of xanthan gum, and an effective amount of anti-obesity agent, together or separately.

14. A product containing the composition of claim 10 as a combined preparation for simultaneous, separate or sequential use in obesity or an obesity-related disorder.

15. A composition comprising
(a) xanthan gum, or a pharmaceutically acceptable salt thereof; and
(b) an anti-obesity agent selected from the group consisting of:
(1) 5HT transporter inhibitor,
(2) NE transporter inhibitor,
(3) CB-1 antagonist/inverse agonist,
(4) Ghrelin antagonist,
(5) H3 antagonist/inverse agonist,
(6) MCH1R antagonist,
(7) MCH2R agonist/antagonist,
(8) MC4R agonist,
(9) NPY1 antagonist,
(10) NPY5 antagonist,
(11) leptin,
(12) leptin derivative,
(13) opioid antagonist,
(14) orexin antagonist,
(15) BRS3 agonist,
(16) CCK-A agonist,
(17) CNTF,
(18) CNTF derivative,
(19) DP-IV inhibitor,
(20) GHS agonist,
(21) 5HT2C agonist;
(22) monoamine reuptake inhibitor,
(23) UCP-1, 2, and 3 activator;
(24) β3 agonist,
(25) thyroid honnone β agonist,
(26) PDE inhibitor,
(27) FAS inhibitor,
(28) DGAT1 inhibitor,
(29) DGAT2 inhibitor,
(30) ACC2 inhibitor,
(31) glucocorticoid antagonist,
(32) acyl-estrogens,
(33) lipase inhibitor;
(34) fatty acid transporter inhibitor,
(35) dicarboxylate transporter inhibitor,
(36) glucose transporter inhibitor, and
(37) serotonin reuptake inhibitor,
or a pharmaceutically acceptable salt thereof.

16. The composition according to claim 15 further comprising a pharmaceutically acceptable carrier.

17. The use of the composition of claim 15 for the manufacture of a medicament for the treatment of obesity or an obesity-related disorder which comprises an effective amount of xanthan gum and an effective amount of anti-obesity agent, together or separately.

18. A product containing the composition of claim 15 as a combined preparation for simultaneous, separate or sequential use in obesity or an obesity-related disorder.
